(19) United States
(12) Patent Application Publication
(10) Pub. No.: US 2024/0102887 A1
(43) Pub. Date: Mar. 28, 2024

(54) METHOD FOR TESTING AN ORIENTATION OF A DISTAL END OF AN OPTICAL FIBER AND TEST DEVICE THEREFOR

(71) Applicant: Erbe Elektromedizin GmbH, Tuebingen (DE)

(72) Inventors: Markus REITERER, Loipersbach (AT); Andreas Hahnekamp, Sigless (AT); Nikolaus Dragostinoff, Wien (AT)

(21) Appl. No.: 18/471,395

(22) Filed: Sep. 21, 2023

(30) Foreign Application Priority Data
Sep. 27, 2022 (EP) 22198067.5

(51) Int. Cl. G01M 11/00; A61B 18/14

(52) U.S. Cl. CPC G01M 11/33; A61B 18/14; A61B 2017/00725

(57) ABSTRACT

A method and a test device are disclosed for testing whether the relative position and orientation of a distal end of an optical fiber relative to its electrode and/or handle piece of a medical instrument is correct. Light is coupled into a proximal end of optical fiber and is emitted at the distal end thereof onto a projection surface. At the proximal end the light of the light source is coupled eccentrically and/or asymmetrically relative to a center of a proximal face of the optical fiber. A light pattern is created on the projection surface that can be tested based on at least one test criterion in order to test the correct arrangement or orientation of the distal end. The test can be carried out automatically by a camera and a central unit or by a user by observing the light pattern on the projection surface.

### CROSS REFERENCE TO RELATED APPLICATION(S)

This application claims the benefit of European Patent Application No. 22198067.5, filed Sep. 27, 2022, which is incorporated herein by reference in its entirety.

### TECHNICAL FIELD

The invention refers to a method for testing an orientation and/or positioning of a distal end of an optical fiber, which is part of a medical instrument. The method is configured to test whether the distal end and particularly a distal face at the distal end of the optical fiber is correctly orientated and/or positioned relative to a handle piece and/or an electrode of the medical instrument. The invention refers in addition to a test device that is configured for carrying out the method.

### BACKGROUND OF THE INVENTION

For example, the method and the test device can at least randomly be used for testing of manufactured medical instruments, in order to guarantee their functionality or sufficient quality. They can in addition or as an alternative also be used to test reusable instruments after use, cleaning or sterilization.

US 2017/0167861 A1 describes a method for testing the orientation of an optical fiber relative to a lens, particularly a GRIN-lens, thereby light is emitted from the fiber through the lens on a projection surface. The fiber is then moved relative to the lens until the desired orientation is reached. This is tested or recognized by centering a light spot on the projection surface.

In the method known from US 2011/0228259 A1 a correct arrangement of one or more optical fibers on a coupling element coupled therewith is tested by means of a sensor. For this purpose, light is emitted via the at least one optical fiber and the coupling element and a sensor and it is then determined whether the emitted light is incident at a correct position on the sensor. In addition, also the intensity of the light incident on the sensor can be tested in order to test the transmission through the arrangement consisting of the at least one optical fiber and the coupling element.

### SUMMARY OF THE INVENTION

Starting from the known prior art, it can be considered as one object of the present invention to improve testing of an orientation of a distal end of an optical fiber of a medical instrument using simple means.

This object is solved by means of a method as well as a test device as disclosed herein.

According to the invention, the orientation and/or positioning of the distal end of an optical fiber of a medical instrument relative to a handle piece and/or relative to an electrode of the medical instrument is tested in order to guarantee its intended function.

The optical fiber of the medical instrument has a distal face at a distal end and a proximal face at the proximal end. During the intended use of the medical instrument, e.g. during an electrosurgical procedure, light can be received at the distal face and can be transmitted via the optical fiber to the proximal end. There the light is at least partly decoupled at the proximal face and can then be evaluated, for example in order to determine or classify the tissue treated with the medical instrument. The orientation and/or positioning of the distal end of the optical fiber is of importance so that sufficient light can be received.

As an option the optical fiber can comprise at least one coating layer that surrounds the core of the optical fiber. The face of the core of the optical fiber without the at least one coating layer is considered as proximal and distal face of the optical fiber respectively.

During the method according to the invention light for testing the orientation of the distal end is guided through the optical fiber. It is coupled in the proximal face by means of a light source. In a first test condition the coupling of light in the proximal face is performed asymmetrically and/or eccentrically relative to a center of the proximal face. The proximal face is, therefore, not regularly irradiated with light. Preferably light is coupled in the proximal face only in a continuous or non-continuous surface section that is non-rotationally symmetrically relative to the center of the proximal face or the geometric center of gravity of the surface section is offset relative to the center of the proximal face. Particularly, the proximal face is not entirely two-dimensionally circularly irradiated with light in a concentrical manner relative to its center. This first test condition is used in order to test the relative position and orientation of the distal end of the optical fiber relative to the handle piece and/or the electrode.

The light coupled in the proximal face is at least partly emitted at the distal face onto a projection surface and creates a light pattern on the projection surface. For example, the light pattern is a closed ring or a ring arc. The distal face is thereby in a defined position or orientation relative to the projection surface. As a result, also the electrode or the handle piece of the medical instrument has a defined position and/or orientation relative to the projection surface. Based on the light pattern created on the projection surface, thus the relative position and orientation of the distal end relative to the handle piece and/or the electrode can be assessed. For this purpose the created light pattern is tested based on at least one predefined test criterion. For example, one or more of the following test criteria can be used:
Comparison of the actual shape or actual geometry of the light pattern with a desired shape or desired geometry;
Comparison of an actual value of at least one dimension parallel to the projection surface with an assigned desired value respectively.

For comparison of desired values with actual values the test criterion can define an allowable deviation between the respective desired value and the respective actual value.

If the evaluation reveals that the light pattern fulfills the at least one test criterion, the distal end of the optical fiber is correctly positioned and/or orientated. Otherwise, the medical instrument does not comply with the requirements and can be repaired and/or sorted out as a result, for example.

The method can be carried out in the context of the production of medical instruments randomly for a part of the manufactured medical instruments or for all manufactured medical instruments.

It is also possible to test a medical instrument after one or multiple uses respectively, in order to determine changes in the relative position or the relative orientation of the distal end of the optical fiber relative to the handle piece and/or the electrode. Such changes can occur, for example, due to deformations and/or plastic changes at connection locations, particularly adhesive bond locations, e.g. as a result of sterilization of the medical instrument.

Optionally by means of the method according to the invention and/or the test device according to the invention it can be switched between the first test condition and an additional, second test condition. This optional second test condition serves to test the fit of the electrode in or on the handle piece or the relative position between the electrode and the handle piece. For this purpose, light is emitted at the distal end of the optical fiber, such that the electrode is located in the light path between the distal face and the projection surface. In doing so, a silhouette of the electrode can be recognized on the projection surface. The position and/or orientation of the silhouette relative to the light pattern on the projection surface can be evaluated in order to check the relative position between the electrode on one hand and the handle piece and the optical fiber on the other hand. Thus, it can be checked whether the electrode is correctly arranged on the handle piece.

In the second test condition a continuous, preferably circular, two-dimensional light pattern can be created on the projection surface. In the second test condition and without consideration of the silhouette of the electrode the light pattern forms a particularly entirely illuminated, preferably circular illuminated area on the projection surface. In the second test condition the light of the light source can be coupled in the proximal face of the optical fiber symmetrically and/or concentrically and/or in the shape of a continuous, preferably circular, two-dimensional light spot relative to a center of the proximal face. Additionally or alternatively, the optical fiber can be arranged between its distal end and its proximal end, so that a mode mixing occurs in the second test condition.

The medical instrument is preferably a monopolar or bipolar electrosurgical instrument that is particularly used for coagulation and/or cutting of human and/or animal tissue of a patient.

As already mentioned, the test criterion or one of the used test criteria can be the test of the deviation between an actual shape and a desired shape of a light pattern. Thereby, as an example, the actual shape and the desired shape can be a circular ring or a circular arc. For example, the actual shape can be tested whether it has the same roundness as the desired shape or the like. In addition or as an alternative, the actual value of an inner diameter and/or the actual value of an outer diameter of the circular ring or the circular arc can be compared with a respectively assigned desired value, for example. In general, one or more actual values of a dimension can be compared with a respectively assigned desired value for this dimension, e.g. also the width or the thickness of a circular ring or circular arc (difference between the outer diameter and the inner diameter).

Preferably the light coupled in the proximal face has a spectrum that comprises one or multiple light wavelengths that can occur during an intended use, e.g. during an electrosurgical procedure on at least one tissue type.

The evaluation of the light pattern can be carried out automatically or by an operating person. In both cases markings can be present on the projection surface that facilitate the use of the at least one test criterion and, as an example, indicate a desired value for a dimension and/or a desired shape of the light pattern.

In a preferred embodiment the light pattern on the projection surface is detected by means of a camera. The projection surface can be provided on a screen, which is at least partly transparent, so that the camera can detect the created light pattern on the back surface of the screen opposite the projection surface. The camera can also be directed on the projection surface. Preferably the optical axis of the camera is arranged orthogonal to the projection surface. The projection surface is particularly a planar surface.

In a preferred embodiment the optical fiber is a multi-mode fiber (MMF). In this case it is advantageous, if the optical fiber is arranged between its distal end and its proximal end so that mode mixing is avoided. For this purpose, the optical fiber is arranged between the distal end and the proximal end in a manner, which is sufficiently stretched or with sufficiently low curvature. Preferably, between the proximal end and the distal end it forms no arcs and/or loops, which are curved so that they would cause mode mixing. For example, the optical fiber can extend along a straight line between the proximal end and the distal end.

The proximal face and/or the distal face can be planar or convex. In both cases a respective face can be a polished surface. A planar face can also be a non-post-treated fractured surface.

In a preferred embodiment a continuous light spot is created by means of the light source in a surface section of the proximal face that is arranged eccentrically relative to the center of the proximal face. The centroid of an area off the light spot or surface section is thereby offset relative to the center of the proximal face. The light spot or surface section can be circular or elliptic, for example. Alternatively to this, the surface section can also be formed by multiple separate circular and/or elliptic light spots. In another embodiment the surface section can also be ring-shaped, concentrically or eccentrically arranged relative to the center of the proximal face. In any case, the surface section, in which light is coupled in the optical fiber, is inhomogeneous radial to the center of the proximal face and/or is inhomogeneous in circumferential direction around the center of the proximal face.

The test device according to the invention comprises a light source, a projection surface as well as a holder. The holder is configured to arrange a component of the medical instrument in a predefined relative position or relative orientation relative to the projection surface, wherein the component is connected with the distal end of the optical fiber. The light source is configured to couple light in the proximal face in an asymmetric and/or eccentric manner. The test device is configured to be able to carry out any embodiment of the method described above.

The evaluation of the light pattern can be carried out automatically or by an operating person. For automatic evaluation the test device can comprise a camera as an option. At least one image of the light pattern captured by the camera can be transmitted to a central unit or evaluation unit. The central unit is in this case configured to automatically evaluate the at least one image of the light pattern based on the at least one test criterion. The central unit can be optionally configured to control the camera and/or the light source.

### BRIEF DESCRIPTION OF THE DRAWINGS

Advantageous embodiments of the invention are derived from the dependent claims, the description and the drawings. In the following, preferred embodiments of the invention are explained in detail based on the attached drawings. The drawings show:

FIG. 1 a schematic illustration of an embodiment of a medical instrument as well as an apparatus to which the medical instrument is connected during operation,

FIG. 2 a block diagram of an embodiment of a test device for the medical instrument for testing the position and/or orientation of an optical fiber relative to a handle piece or an electrode of the medical instrument,

FIG. 3 a schematic principal illustration of a distal face of the optical fiber of the medical instrument from FIGS. 1 and 2 as well as a surface section in which light is coupled in the proximal face of the optical fiber in a first test condition and

FIG. 4 a schematic principal illustration of a light pattern created on a projection surface of the test device in a first test condition,

FIG. 5 a schematic principal illustration of the distal face of the optical fiber of the medical instrument of FIGS. 1 to 3 as well as the surface section in which light is coupled in the proximal face of the optical fiber in a second test condition and

FIG. 6 a schematic principal illustration of a light pattern created on a projection surface of the test device in a second test condition.

### DETAILED DESCRIPTION

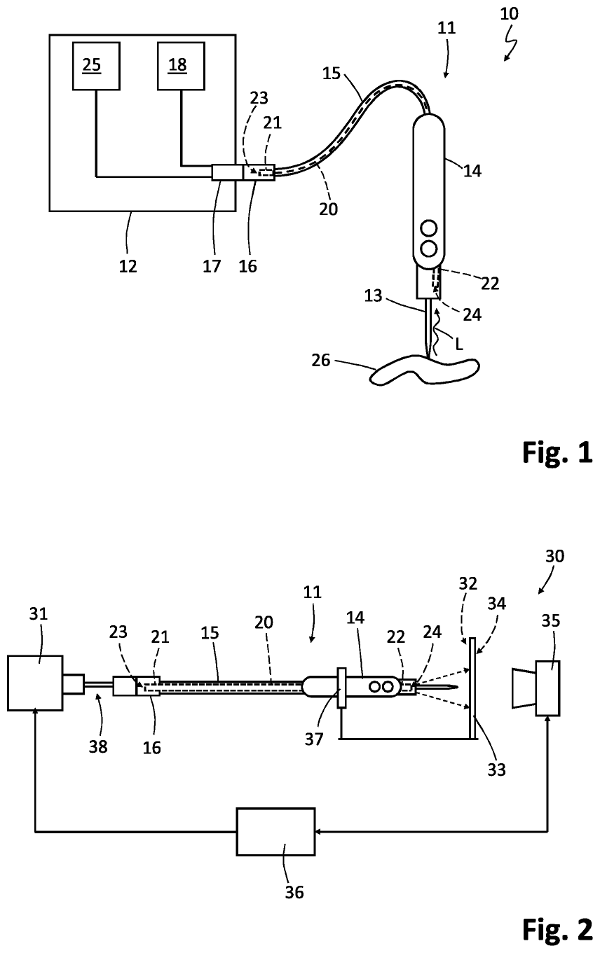

FIG. 1 shows a system 10 comprising a medical instrument 11 as well as an apparatus 12 to which the medical instrument 11 can be connected. The medical instrument 11 is an electrosurgical instrument and can be configured as monopolar or bipolar medical instrument 11 having at least one electrode 13. The electrode 13 is arranged on a handle piece 14 of the medical instrument 11.

A cable 15 extends from the handle piece 14 with the electrode 13 to a connection element 16. A connection 17 is provided on apparatus 12 to which the connection element 16 can be connected. According to the example, by means of the connection element 16 and the connection 17 at least one electrical as well as an optical connection is established between apparatus 12 and medical instrument 11.

With the connection established between medical instrument 11 and apparatus 12 the electrode 13 is connected to an electrical energy source 18 of apparatus 12 via at least one electrical conductor. The at least one electrical conductor is part of the cable 15 and not illustrated in the drawing.

In addition, in the cable 15 an optical fiber 20 extends from a proximal end 21 at connection element 16 to a distal end 22 at handle piece 14. Relative to the handle piece 14 the distal end 22 is immovably attached to the handle piece 14, preferably by means of a substance bond connection, particularly an adhesive connection. In addition or as an alternative, the distal end 22 can also be connected to handle piece 14 in a force-fit and/or form-fit manner.

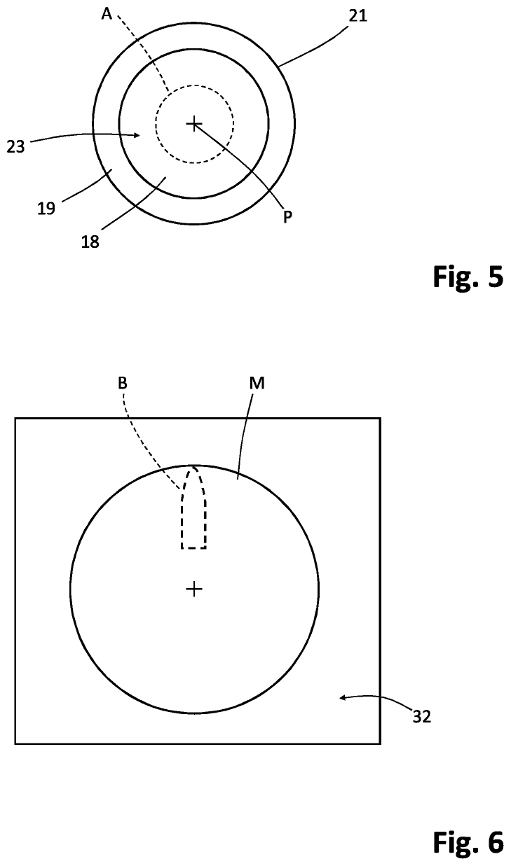

The optical fiber 20 comprises a proximal face 23 at the proximal end 21 and the optical fiber 20 comprises a distal face 24 at the distal end 22. The optical fiber 20 can have a core 18 and a coating 19 (FIGS. 3 and 5). According to the example, the faces of the core 18 without the coating 19 are considered as proximal face 23 and distal face 24.

With the connection established, an optical connection is established between the proximal end 21 and an evaluation unit 25 of apparatus 12 by means of connection element 16 and connection 17.

According to the example, medical instrument 11 is configured as an electrosurgical instrument, whereby animal or human tissue 26 can be treated by means of the at least one electrode 13, e.g. by coagulation and/or cutting. Light L created during treatment of tissue 26 is received at distal face 24 and is guided via the optical fiber 20 to the proximal end 21. At the proximal end 21 light L is at least partly further transmitted to evaluation unit 25 via connection 17 and can be evaluated there, e.g. in order to classify and/or determine the tissue 26.

In order to be able to carry out such an evaluation of the treated tissue 26, optical fiber 20 and particularly its distal end 22 have to be very precisely arranged and/or orientated relative to the handle piece 14 or relative to electrode 13, in order to capture a sufficient amount of light L that is created during treatment of tissue 26. Otherwise, an evaluation or classification of tissue 26 is not possible or only in an insufficient manner.

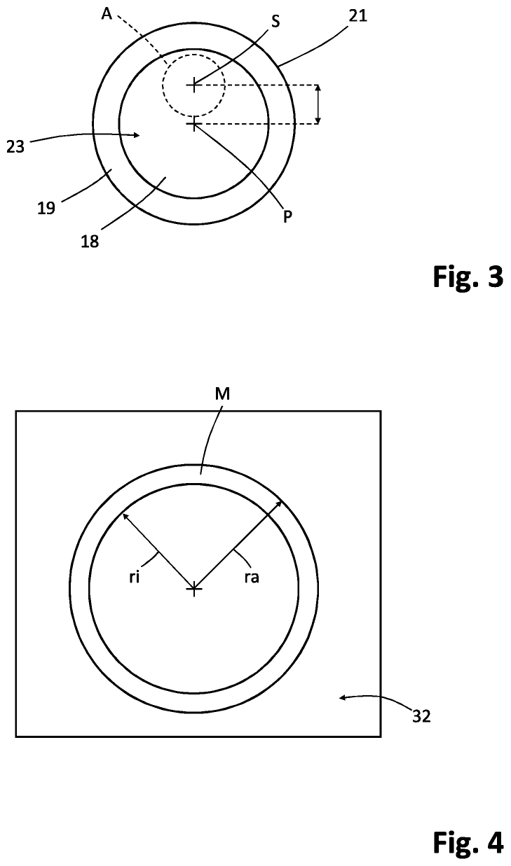

According to the invention, therefore, medical instrument 11 is tested whether the orientation and/or positioning of distal end 22 of optical fiber 20 relative to handle piece 14 and/or the at least one electrode 13 is correct and complies with the requirements. For this purpose, a test device 30 can be used, as is schematically depicted in FIG. 2. The test device 30 comprises a light source 31 for coupling of light into the proximal face 23 of optical fiber 20 as well as a projection surface 32. The projection surface 32 is arranged and orientated so that light exiting the distal face 24 of optical fiber 20 impinges on projection surface 32 and creates a light pattern M there (FIG. 4). The light pattern M can be a circular ring, for example, as shown in FIG. 4, or alternatively also only a section thereof, i.e. a circular arc.

According to the example, projection surface 32 is a planar surface. In the embodiment projection surface 32 is present on a screen 33 of the test device. For example, projection surface 32 can be arranged orthogonal to the extension direction of electrode 13 and can be arranged near a free end of the electrode 13 or can be in contact therewith.

On the backside 34 of screen 33 opposite the projection surface 32 a camera 35 of test device 30 is arranged as an option. The screen 33 is at least partly transparent for the light of light source 31, so that the camera 35 is able to capture on the backside 34 the light pattern M created on the projection surface 32. The camera 35 is communicatively connected with a central unit 36. An image of the light pattern M captured by camera 35 can be transmitted to the central unit 36. The central unit 36 can be configured to control the light source 31 and the camera 35. By means of a holder 37 of the test device 30, handle piece 14 is put in a defined position and/or orientation relative to the projection surface 32 according to the example. By means of holder 37, thus also the relative position and relative orientation of distal face 24 of the optical fiber 20 is defined relative to the projection surface 32.

In the embodiment optical fiber 20 is a multi-mode fiber (MMF). According to the example, proximal face 23 and distal face 24 are configured as planar surfaces and could in modification thereto also be curved convexly.

For example, light source 31 can be a semi-conductor light source and can create light by means of a light emitting diode or laser diode.

In a test condition that is denoted as first test condition for sake of distinction compared to an optional additional test condition, light of light source 31 is coupled into proximal end 21 of optical fiber 20 asymmetrically and/or eccentrically relative to a center P of proximal face 23, as highly schematically apparent in FIGS. 2 and 3. In an embodiment described here, light of light source 31 is coupled into a surface section A of proximal face 23, wherein surface section A can have a circular or elliptical shape, for example. The surface section A has a centroid of an area S. The centroid of an area S is arranged with distance to the center P of proximal face 23, as schematically depicted in FIG. 3. Alternatively to the illustration in FIG. 3, the center P can also be arranged inside surface section A into which light of the light source 31 is irradiated. In any case surface section A, into which light of the light source 31 enters, is selected so that light of the light source 31 is irregularly coupled into proximal face 23 radially to the center P and/or in circumferential direction around center P.

In modification to the example shown in FIG. 3 the surface section A can be non-continuous, so that light of light source 31 can be coupled in proximal face 23 via multiple portions of surface section A that are distanced from one another.

As schematically illustrated in FIG. 2, a coupling device 38 can be part of test device 30, which is configured for optical coupling of optical fiber 20 with light source 31. The coupling device 38 can be configured, for example, to establish an optical connection between the connection element 16 and the light source 31, so that light of the light source 31 is coupled in asymmetrically and/or eccentrically relative to the center P of proximal face 23—as described above.

In order to avoid mode mixing during test in the first test condition, optical fiber 20 is preferably aligned sufficiently straight during the test and can extend substantially linearly between proximal end 21 and distal end 22, as schematically depicted in FIG. 2. In modification thereto optical fiber 20 could also extend in minor arcs having a sufficiently small curvature, in order to avoid mode mixing.

Due to coupling light of light source 31 into the proximal face 23, asymmetrically and/or eccentrically in the first test condition, the light exiting the distal face 24 creates the light pattern M on the projection surface 32, according to the example a light pattern M in the shape of a closed circular ring having an outer radius ra and an inner radius ri (FIG. 4).

Based on the shape of the light pattern M and/or at least one dimension of the light pattern M in at least one spatial direction parallel to the projection surface 32, it can be tested in the first test condition whether the relative positions of relative orientation of distal end 22 or distal face 24 on one hand relative to electrode 13 and/or handle piece 14 on the other hand comply with the requirements. For example, it can be tested whether the actual shape of light pattern M corresponds to a desired shape of light pattern M, thus, for example, whether light pattern M comprises a circular ring shape or a circular arc shape. Additionally or alternatively, also one or multiple parameters can be tested, such as an actual value for the inner radius ri and/or an actual value for the outer radius ra, which can be respectively compared with an assigned desired value.

By using at least one test criterion in the first test condition it can be determined whether medical instrument 11 and particularly the attachment of optical fiber 20 to handle piece 14 complies sufficiently well with the requirements and therefore the medical instrument 11 fulfills the quality requirements.

The first test condition (FIGS. 2 to 4) can be the sole test condition of the method and/or the test device 30. Optionally an additional, second test condition of the method and/or test device 30 can be provided (FIGS. 5 and 6), wherein it is possible to switch between first test condition and second test condition. This optional second test condition serves to test the fit of electrode 13 in the handle piece 14 or the relative position between the electrode 13 and the handle piece 14. For this purpose, light is emitted from the distal end 22 of the optical fiber so that the electrode 13 is located in the light path between the light exiting from the distal face 24 and the projection surface 32. In doing so, a silhouette B of the electrode is visible on the projection surface 32 (FIG. 6). The position of silhouette B relative to light pattern M on the projection surface 32 can be evaluated in order to test the relative position between electrode 13 on one hand and handle piece 14 and optical fiber 20 on the other hand. Thus, it can be tested whether electrode 13 is correctly arranged on handle piece 14.

According to the example, a continuous two-dimensional light pattern M is created on the projection surface 32 in the second test condition (FIG. 6). Without consideration of silhouette B of electrode 13, the light pattern M forms an entirely illuminated circular light area on the projection surface 32 in the second test condition, as an example. In the second test condition the light of light source 31 can be coupled asymmetrically and/or concentrically and/or in the shape of an entirely illuminated continuous—preferably circular-shaped—light spot relative to center P of proximal face 23 at the proximal face 23 of optical fiber 20. Additionally or alternatively, the optical fiber 20 can be arranged between its distal end 22 and its proximal end 21 in the second test condition, so that mode mixing occurs. For example, optical fiber 20 can be arranged in a manner deviating from its stretched orientation so as to be curved or bent at least once with sufficient amount.

For example, for newly manufactured medical instruments 11 the test in the first and/or second test condition can be carried out randomly or for all manufactured medical instruments 11. In addition, the possibility exists to test a medical instrument 11 repeatedly after its use and/or sterilization so that it can be determined, if due to external influences, particularly thermal influences during sterilization, modifications such as plastic deformations or the like occur that result in the medical instrument 11 no longer complying with the requirements. For example, due to thermal influences the adhesive bond between the distal end 22 of optical fiber 20 and the handle piece 14 can be affected, whereby the orientation or position of the distal end 22 relative to the handle piece 14 may change.

The invention refers to a method and a test device 30 in order to test whether the relative position and relative orientation of a distal end 22 of an optical fiber 20 relative to its electrode 13 and/or handle piece 14 of a medical instrument 11 comply with the requirements and is thus correct. For this purpose, light is coupled into a proximal end 21 of optical fiber 20 and is emitted at the distal end 22 of optical fiber 20 onto a projection surface 32. At the proximal end 21 the light of the light source 31 is coupled in eccentrically and/or asymmetrically relative to a center P of a proximal face 23 of optical fiber 20. In doing so, a light pattern M is created on projection surface 32, particularly a ring-shaped light pattern M that can be tested based on at least one test criterion in order to test the correct arrangement or orientation of the distal end 22. The test can be carried out automatically by means of a camera 35 and a central unit 36 or by an operating person by means of observation of the light pattern M on the projection surface 32. Optionally in another test condition it can be tested whether the electrode 13 is correctly arranged on the handle piece 14.

### LIST OF REFERENCE SIGNS

10 system
11 medical instrument
12 apparatus
13 electrode
14 handle piece
15 cable
16 connection element
17 connection
18 core
19 coating
20 optical fiber
21 proximal end
22 distal end
23 proximal face
24 distal face
25 evaluation unit
26 tissue
30 test device
31 light source
32 projection surface
33 screen
34 backside
35 camera
36 central unit
37 holder
38 coupling device
A surface section
B silhouette
L light
M light pattern
P center
ra outer radius
ri inner radius
S centroid of an area

1. A method for testing an orientation and/or positioning of a distal end (22) of an optical fiber (20) of a medical instrument (11) relative to a handle piece (14) of the medical instrument (11) and/or an electrode (13) of the medical instrument (11), the method comprising:
arranging a distal face (24) of the distal end (22) of the optical fiber (20) in an orientation relative to a projection surface (32);
coupling light of a light source (31) into a proximal face (23) at a proximal end (21) of the optical fiber (20) opposite the distal end (22) in an asymmetric and/or eccentric manner relative to a center (P) of the proximal face (23);
emitting at least a part of the coupled light from the distal face (24) at the distal end (22) of the optical fiber (20) onto the projection surface; and
evaluating a light pattern (M) created on the projection surface (32) based on at least one test criterion for testing the orientation of the distal end (22) of the optical fiber (20).

2. The method according to claim 1, wherein the at least one test criterion comprises a test of a deviation between an actual shape of the light pattern (M) on the projection surface (32) and a desired shape of the light pattern (M).

3. The method according to claim 2, wherein the actual shape of the light pattern (M) and/or the desired shape of the light pattern (M) is a closed ring or ring arc.

4. The method according to claim 1, wherein the at least one test criterion comprises a test of a deviation between an actual value of a dimension of the light pattern (M) in at least one spatial direction parallel to the projection surface (32) and a desired value of the dimension of the light pattern (M) in the at least one spatial direction.

5. The method according to claim 1, wherein an actual shape of the light pattern (M) and/or a desired shape is a closed ring or ring arc and the at least one test criterion comprises a test of a deviation between a desired value of the dimension of an inner radius (ri) and/or an outer radius (ra) of the closed ring or ring arc and an actual value of the dimension of the inner radius (ri) and/or the outer radius (ra) of the closed ring or ring arc.

6. The method according to claim 1, further comprising capturing an image of the light pattern (M) on the projection surface (32) by a camera (35) and transmitting the image to a central unit (36) for automatic evaluation based on the at least one test criterion.

7. The method according to claim 6, wherein the central unit (36) is configured to control the camera (35) and the light source (31).

8. The method according to claim 1, wherein the optical fiber (20) is a multi-mode fiber.

9. The method according to claim 7, wherein the optical fiber (20) is arranged between its distal end (22) and its proximal end (21) so that mode mixing is avoided.

10. The method according to claim 8, wherein the optical fiber (20) is substantially stretched between its distal end (22) and its proximal end (21).

11. The method according to claim 1, wherein the light of the light source (31) is coupled into a surface section (A) of the proximal face (23) of the optical fiber (20), wherein a centroid of an area (S) of the surface section (A) is offset relative to the center (P) of the proximal face (23).

12. The method according to claim 1, wherein the proximal face (23) and/or the distal face (24) is a planar surface.

13. The method according to claim 1, further comprising, in another test condition, illuminating the electrode (13) by light exiting the distal face (24) in order to create a silhouette (B) of the electrode (13) on the projection surface (32) to test based on the silhouette (B) whether the electrode (13) is correctly arranged relative to the handle piece (14).

14. A test device (30) for testing an orientation and/or positioning of a distal end (22) of an optical fiber (20) of a medical instrument (11) relative to a handle piece (14) of the medical instrument (11) and/or an electrode (13) of the medical instrument, the test device (30) comprising:
a light source (31) configured for coupling light into a proximal face (23) at a proximal end (21) of the optical fiber (20) asymmetrically and/or eccentrically relative to a center (P) of the proximal face (23),
a projection surface (32) arranged and oriented so that light from the light source (31) exiting a distal face (24) of the optical fiber (20) impinges on the projection surface (32), and
a holder (37) configured for holding the handle piece of the medical instrument such that a distal end (22) of the optical fiber (20) is arranged in a relative position relative to the projection surface (32) to allow at least a part of the coupled light to be emitted from the distal face (24) of the optical fiber (20) onto the projection surface (32) to create a light pattern (M) thereon.

15. The test device according to claim 14, further comprising a camera (35) for capturing an image of the light pattern (M) created on the projection surface (32).
